(19) United States
(12) Patent Application Publication
(10) Pub. No.: US 2010/0152284 A1
(43) Pub. Date: Jun. 17, 2010

(54) PREVENTION AND REVERSAL OF CHEMOTHERAPY-INDUCED PERIPHERAL NEUROPATHY

(75) Inventors: David Brown, New South Wales (AU); Alan James Husband, New South Wales (AU); Ann M. Turnley, Victoria (AU)

Correspondence Address: MCHALE & SLAVIN, P.A., 2855 PGA BLVD, PALM BEACH GARDENS, FL, 33410, US

(73) Assignee: NOVOGEN RESEARCH PTY LTD, North Ryde NSW (AU)

(21) Appl. No.: 12/447,624

(22) PCT Filed: Oct. 30, 2007
(86) PCT No.: PCT/AU2007/001643

§ 371 Date: Mar. 4, 2010

(30) Foreign Application Priority Data
Oct. 30, 2006 (AU) 2006906016

(51) Int. Cl. A61K 31/352; C07D 311/58; A61P 35/00; A61P 25/02

(52) U.S. Cl. 514/456; 549/406

(57) ABSTRACT

Provided herein are methods for treating or preventing neuropathy, neuropathy-related conditions, wherein the neuropathy or neuropathy-related conditions are induced by, or otherwise associated with, treatment of the subject with at least one chemotherapeutic agent, the methods comprising administering an effective amount of an isoflavonoid compound of formula (I). Also provided are methods for the treatment of nerve damage. Also provided are uses of isoflavonoid compounds of formula (I) in the treatment of neuropathy, neuropathy-related conditions and nerve damage.

### FIELD OF THE INVENTION

The present invention relates generally to the treatment, prevention or reversal of neuropathy and related conditions, in particular to peripheral neuropathy induced by chemotherapy. More particularly, the present invention relates to the use of isoflavonoids as described herein for such treatment, prevention or reversal.

### BACKGROUND OF THE INVENTION

Peripheral neuropathy is a condition of the peripheral nervous system in which damage to peripheral nerves can cause severe pain and a range of symptoms in sufferers including numbness, tingling sensations, burning sensations, parasthesia and muscle weakness in various parts of the body. In severe cases, peripheral neuropathy can result in paralysis and organ or gland dysfunction.

Peripheral neuropathy can be caused by a range of factors including as a result of infectious agents such as viruses, inflammatory conditions, or exposure to neurotoxic compounds. Peripheral neuropathy can also result as a side-effect of drug treatment regimens, for example, anti-HIV drugs and chemotherapeutic agents. Indeed many commonly-employed chemotherapeutic agents are limited in their effectiveness due to side-effects such as peripheral neuropathy. This is particularly problematic for use of otherwise highly effective anti-neoplastic agents, such as platinum analogues or taxane family members, as the effects are often dose-limiting (see, for example, Macdonald, 1991; Quasthoff and Hartung, 2002).

The platinum analogue cisplatin (Cis-diamine-dichloro-platinum) has been used as a chemotherapeutic for nearly 40 years and is one of the most widely-used cytotoxic drugs. Cisplatin produces its anti-neoplastic effects by binding directly to DNA, resulting in cross-linking and production of apoptosis in rapidly dividing cells. Cisplatin is also taken up directly by post-mitotic sensory neurons, and is known to produce neuropathy. Neuropathies normally develop after prolonged (typically at least 4 months) cisplatin therapy, but have been reported after single administrations, it is presently recommended that cisplatin therapy be ceased when symptoms of neuropathy are first encountered (ref). Severe neuropathy may be experienced in approximately 4% of patients receiving cisplatin therapy, while mild neuropathy may be experienced by around 40% of patients. The mechanism by which cisplatin induces neuropathy is unclear. While it does appear to induce apoptosis in sensory neurons, early stages involve axonal loss but not necessarily cell loss and it has been proposed to involve a disturbance of cytoplasmic/axonal transport. The related chemotherapeutic agents carboplatin and oxaliplatin have also been reported to cause neuropathy.

Microtubules are required for mitosis and other vital functions. The taxane derivative paclitaxel (Taxol) is a highly effective chemotherapeutic agent that promotes the polymerization of tubulin, thereby aggregating microtubules and promoting cell death by inhibiting their normal activity. Neurite outgrowth is also very reliant on normal microtubule function and numerous studies have shown the effects of paclitaxel on abnormal microtubule formation in neurites and inhibition of neurite outgrowth (Letourneau and Ressler, 1984). However the major limiting side effect of paclitaxel is neurotoxicity which, as a result of cumulative effects, is observed in most patients. As with cisplatin, the mechanism by which paclitaxel induces neuropathy is not fully elucidated, although paclitaxel has been shown to induce axonal loss and to disrupt cytoplasmic flow in neurons, with microtubule accumulation in axons. Similar to cisplatin, severe neuropathy is reported to be experienced by approximately 4% of patients receiving paclitaxel therapy, while up to 60% of patients may experience mild symptoms. The effect is typically dose dependent. The related compound docetaxel (Taxotere) is reported to result in slightly higher frequencies of neuropathy.

Other chemotherapeutic agents reported to cause neuropathy include the Vinca alkaloids such as vincristine and vinorelbine (Navelbine), hycamtin (Topotecan), hexamethylmelamine (Hexalen), bortezomib (Velcade), cytarabine and procarbazine. In all cases, the chemotherapeutic treatment is typically ceased, or dosage of the agent reduced, when patients experience symptoms of neuropathy, thereby requiring alternative treatment regimes to be found.

The neurotoxic effects following treatment with chemotherapeutic agents can be severe and significantly affect a patient's quality of life, even long after the treatment has ceased. Although some nerve regeneration may occur, this is often slow and in many instances the reversal of neuropathy is incomplete. Hence the neuropathy can affect quality of life and retard normal nervous system functioning for many years.

Accordingly, there is a clear need for effective strategies to treat or prevent chemotherapy-induced neuropathy or reverse existing neuropathies.

There is currently no treatment strategy available to effectively combat neuropathy caused by any factor, including chemotherapeutic agents. A variety of compounds including neurotrophic or neuroprotective factors, such as nerve growth factor (NGF), insulin-like growth factor-1 (IGF1), erythropoietin and leukaemia inhibitory factor (LIF) have been employed in attempts to inhibit or reverse chemotherapy-induced neuropathies. However all have met with limited success and their clinical use is limited due to difficulties in drug administration, stability, deleterious side effects or ineffectiveness in human clinical trials. Antioxidants such as glutathione and vitamin E, and neuroprotective compounds such as acetyl-L-carnitine have also shown some effectiveness in protecting against chemotherapy-induced neuropathy in preliminary studies. However to date, no compounds capable of reliably preventing or reversing chemotherapy-induced neuropathies have been identified.

### SUMMARY OF THE INVENTION

The present invention is predicated on the inventors' surprising finding that the isoflavanoid phenoxodiol is able to act as a neuroprotective agent, protecting against chemotherapy-induced neuropathy.

Accordingly, a first aspect of the present invention provides a method for treating or preventing neuropathy or a neuropathy-related condition in a subject, wherein the neuropathy or neuropathy-related condition is induced by, or otherwise associated with, treatment of the subject with at least one chemotherapeutic agent, the method comprising administering to the subject an effective amount of an isoflavonoid compound of formula (I):

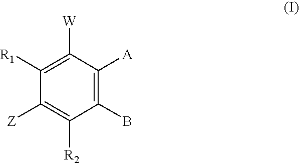

in which
R₁, R₂ and Z are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₂ is as previously defined, and R₁ and Z taken together with the carbon atoms to which they are attached form a five-membered ring selected from

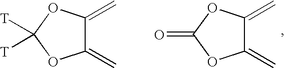

or
R₁ is as previously defined, and R₂ and Z taken together with the carbon atoms to which they are attached form a five-membered ring selected from

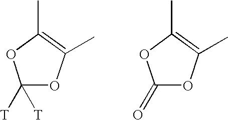

and
W is R₁, A is hydrogen, hydroxy, NR₃R₄ or thio; and B is selected from

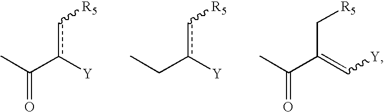

or
W is R₁, and A and B taken together with the carbon atoms to which they are attached form a six-membered ring selected from

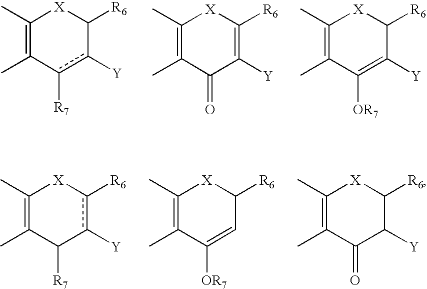

or
W, A and B taken together with the groups to which they are associated are selected from

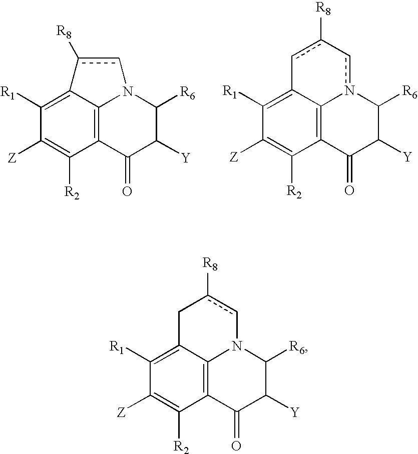

or
W and A taken together with the groups to which they are associated are selected from

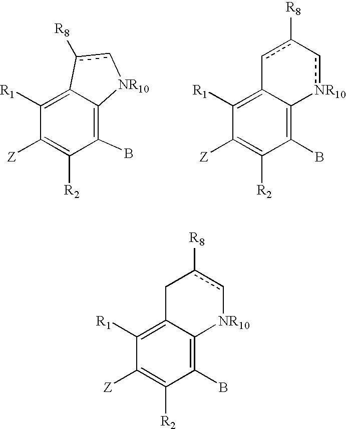

and B is selected from

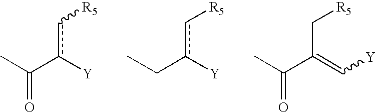

wherein
R₃ is hydrogen, alkyl, arylalkyl, alkenyl, aryl, an amino acid, C(O)R₁₁ where R₁₁ is hydrogen, alkyl, aryl, arylalkyl or an amino acid, or CO₂R₁₂ where R₁₂ is hydrogen, alkyl, haloalkyl, aryl or arylalkyl,
R₄ is hydrogen, alkyl or aryl, or
R₃ and R₄ taken together with the nitrogen to which they are attached comprise pyrrolidinyl or piperidinyl,
R₅ is hydrogen, C(O)R₁₁ where R₁₁ is as previously defined, or CO₂R₁₂ where R₁₂ is as previously defined,
R₆ is hydrogen, hydroxy, alkyl, aryl, amino, thio, NR₃R₄, COR₁₁, where R₁₁ is as previously defined, CO₂R₁₂ where R₁₂ is as previously defined or CONR₃R₄,
R₇ is hydrogen, C(O)R₁₁ where R₁₁ is as previously defined, alkyl, haloalkyl, alkenyl, aryl, arylalkyl or Si(R₁₃)₃ where each R₁₃ is independently hydrogen, alkyl or aryl,
R₈ is hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀ is hydrogen, alkyl, haloalkyl, amino, aryl, arylalkyl, an amino acid, alkylamino or dialkylamino, the drawing [symbol] represents either a single bond or a double bond,
T is independently hydrogen, alkyl or aryl,
X is O, NR₄ or S, and
Y is

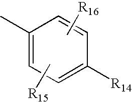

wherein
R₁₀, R₁₅ and R₁₆ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, thio, alkylthio, amino, alkylamino, dialkylamino, nitro or halo, or any two of R₁₄, R₁₅ and R₁₆ are fused together to form a cyclic alkyl, aromatic or heteroaromatic structure, and pharmaceutically acceptable salts thereof.

The chemotherapeutic agent may be any agent used in the treatment of cancer or tumours, wherein administration of the agent causes nerve dysfunction and/or damage, typically peripheral nerves. For example, the chemotherapeutic agent may be selected from the group consisting of, but not limited to: cisplatin, carboplatin, paclitaxel, docetaxel, vincristine, vinorelbine, hycamtin, hexamethylmelamine, bortezomib, cytarabine and procarbazine, and analogues or derivatives thereof. In one embodiment the chemotherapeutic agent is cisplatin or an analogue or derivative thereof.

The isoflavonoid may be administered prior to, during or after administration of the chemotherapeutic agent. The isoflavonoid may be administered in conjunction with the chemotherapeutic agent. The isoflavonoid may be administered via the same route as the chemotherapeutic agent, or by any alternative suitable route.

The isoflavonoid may be selected from the group consisting of:

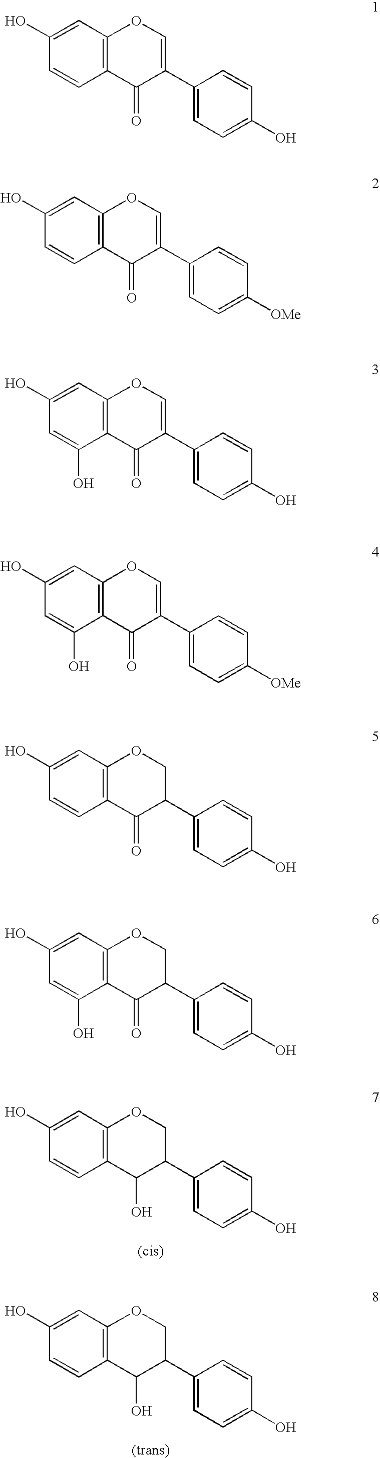

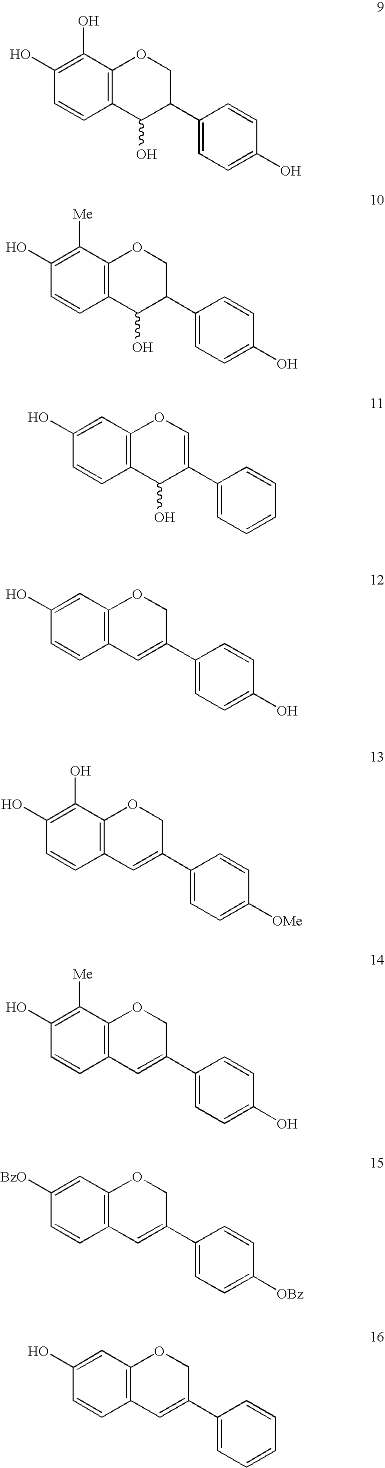

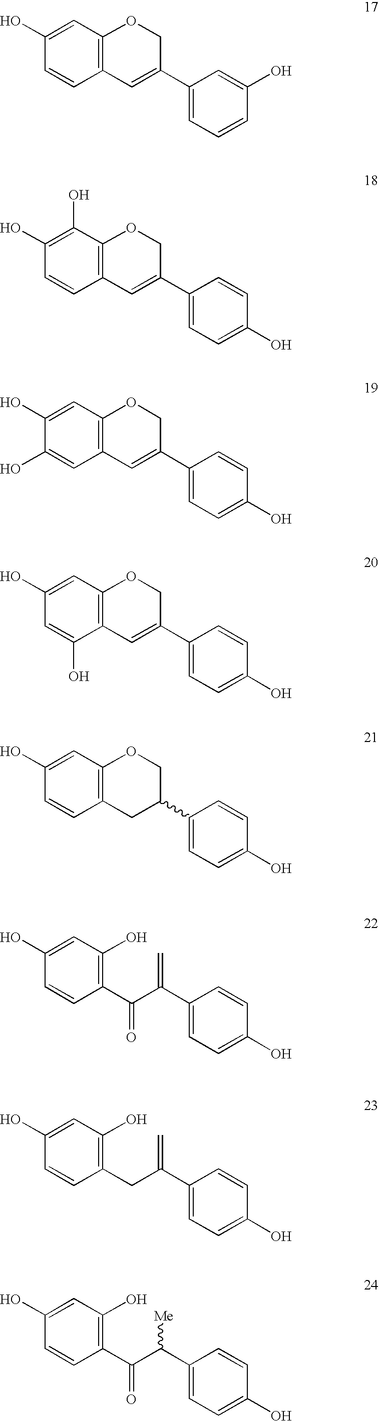

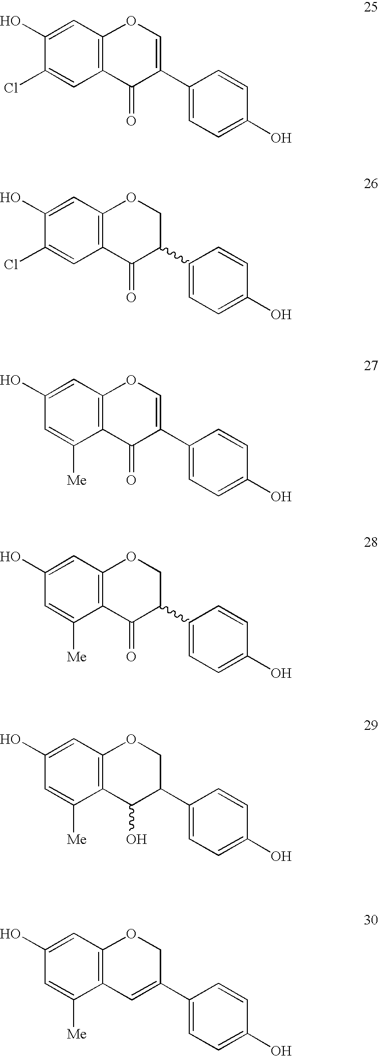

In one embodiment the isoflavonoid is phenoxodiol (compound 12).

According to a second aspect of the invention there is provided a method for treating or preventing nerve damage in a subject, the method comprising administering to the subject an effective amount of an isoflavonoid of formula (I).

The nerve damage may be peripheral nerve damage and is typically induced by, or associated with treatment of the subject with at least one chemotherapeutic agent.

In one embodiment the isoflavonoid is phenoxodiol.

According to a third aspect of the present invention there is provided the use of an isoflavonoid of formula (I) as a neuroprotective agent.

In one embodiment the isoflavonoid is phenoxodiol.

According to a fourth aspect of the present invention there is provided a method for the treatment of cancer in a subject, the method comprising administering to the subject:
(i) a chemotherapeutic agent which has a neurotoxic effect on peripheral nerves, the chemotherapeutic agent being administered at a therapeutically effective dose; and
(ii) an isoflavonoid of formula (I) at a dose effective to prevent, reduce, eliminate or reverse the neurotoxic effect of the chemotherapeutic agent of (i).

The chemotherapeutic agent and the isoflavonoid may be administered concurrently or sequentially.

The neurotoxic effect may be neuronal dysfunction or damage.

In one embodiment the isoflavonoid is phenoxodiol.

According to a fifth aspect of the present invention there is provided the use of an isoflavonoid of formula (I) for the manufacture of a medicament for the treatment or prevention of neuropathy or a neuropathy-related condition, wherein the neuropathy or neuropathy-related condition is induced by, or otherwise associated with, at least one chemotherapeutic agent.

According to a sixth aspect of the present invention there is provided the use of an isoflavonoid of formula (I) for the manufacture of a medicament for the treatment or prevention of nerve damage, wherein the nerve damage is typically induced by, or associated with, a chemotherapeutic agent.

According to a seventh aspect of the present invention there is provided a composition comprising an isoflavonoid of formula (I) when used for the treatment or prevention of neuropathy or a neuropathy-related condition, wherein the neuropathy or neuropathy-related condition is induced by, or otherwise associated with, at least one chemotherapeutic agent.

According to an eighth aspect of the present invention there is provided a composition comprising an isoflavonoid of formula (I) when used for the treatment or prevention of nerve damage, wherein the nerve damage is typically induced by, or associated with, a chemotherapeutic agent.

Typically in accordance with the above aspects and embodiments the subject is human. In other embodiments, the subject may be selected from the group consisting of, but not limited to: primate, ovine, bovine, canine, feline, porcine, equine and murine.

### BRIEF DESCRIPTION OF THE DRAWINGS

The present invention will now be described, by way of non-limiting example only, with reference to the accompanying drawings in which:

FIG. 1 illustrates dose responsiveness of neurite toxicity induced by paclitaxel and cisplatin. The percentage of differentiated PC12 cells with neurites was counted after incubation for 24 hrs in increasing concentrations of (A) paclitaxel (* p<0.001 compared to control) and (B) cisplatin (* p<0.001 compared to control). Both agents showed a dose response in inhibition of neurite outgrowth and moderate and strong concentrations of each (indicated by arrows), were chosen for subsequent analyses.

FIG. 2 demonstrates protection against cisplatin- and paclitaxel-induced neurite toxicity by phenoxodiol. Differentiated PC12 cells were incubated with cisplatin or paclitaxel alone or in combination with phenoxodiol for 24 hrs and the percentage of cells with neurites determined. (A) PC12 cells incubated with phenoxodiol (PXD) alone. (B) PC12 cells incubated with cisplatin (Cis) alone or in combination with phenoxodiol. (C) PC12 cells incubated with paclitaxel (Pac) alone or in combination with phenoxodiol.

FIG. 3 illustrates the effect of cisplatin, paclitaxel and phenoxodiol on neurite length. Differentiated PC12 cells were incubated with Cisplatin or Paclitaxel alone or in combination with PXD for 24 hrs and neurite length determined by measuring the longest neurite on cells with neurites longer than 10 μm. (A) PC12 cells incubated with phenoxodiol (PXD) alone. (B) PC12 cells incubated with cisplatin (Cis) alone or in combination with phenoxodiol. (C) PC12 cells incubated with paclitaxel (Pac) alone or in combination with phenoxodiol.

FIG. 4 shows the effect of phenoxodiol and cisplatin on neurite growth in βIII-tubulin stained PC12 cells. Differentiated PC12 cells were incubated with phenoxodiol (PXD) (B-D), cisplatin (E) and cisplatin in the presence of phenoxodiol (F) for 24 hrs then fixed and immunostained for the neuronal marker βIII-tubulin. Scale bar in (F) represents 50 μm and applies to all panels in the figure.

FIG. 5 shows the effect of phenoxodiol on neurite toxicity in cells pre-treated with cisplatin. Differentiated PC12 cells were incubated with (A) phenoxodiol (PXD) alone or (B) cisplatin (Cis) alone or in combination with phenoxodiol for 24 hrs. The cells were washed and left for 24 hrs, then phenoxodiol at the concentrations indicated was added for a further 24 hrs before the percentage of cells with neurites was determined. Note: the concentrations of phenoxodiol used were 1 log lower than in the previous figures.

### DETAILED DESCRIPTION OF THE INVENTION

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The articles “a” and “an” are used herein to refer to one or to more than one (i.e., to at least one) of the grammatical object of the article. By way of example, “an element” means one element or more than one element.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery. Similarly, in the present context, treatment also includes within its scope the reversal of existing nerve damage or neuropathy, but not necessarily the complete reversal thereof to normal levels that would be expected in the absence of such nerve damage or neuropathy having occurred.

The term “neuropathy-associated condition” as used herein refers to a condition associated with, at least in part, nerve damage, in particular to neurons of the peripheral nervous system. The condition may be characterized by such damage, may occur as a result, either directly or indirectly, of such damage or itself lead to such nerve damage. Typically a “neuropathy-associated condition” will share at least one symptom in common with neuropathy, typically peripheral neuropathy. Such symptoms include loss of sensation, including numbness, tingling or burning sensations in limbs or body extremities, parasthesia, muscle weakness, or a reduction in neuromuscular reflex.

As used herein the terms “effective amount” and “effective dose” include within their meaning a non-toxic but sufficient amount or dose of an agent or compound to provide the desired effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount” or “effective dose”. However, for any given case, an appropriate “effective amount” or “effective dose” may be determined by one of ordinary skill in the art using only routine experimentation.

The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, citrate, bicarbonate and carbonate.

The term “pharmaceutically acceptable derivative” or “prodrug” refers to a derivative of the active compound that upon administration to the recipient, is capable of providing directly or indirectly, the parent compound or metabolite, or that exhibits activity itself. Prodrugs are included within the scope of the present invention.

The terms “isoflavonoid”, “isoflavone” and “isoflavone derivative” as used herein are to be taken broadly to include ring-fused benzopyran molecules having a pendent phenyl group from the pyran ring based on a 1,2-diphenylpropane system. Thus, the classes of compounds generally referred to as isoflavones, isoflavenes, isoflavans, isoflavanones, isoflavanols and the like are generically referred to herein as isoflavones, isoflavone derivatives or isoflavonoid compounds.

The term “alkyl” is taken to mean both straight chain and branched chain alkyl groups such as methyl, ethyl, propyl, isopropyl, butyl, isobutyl, secbutyl, tertiary butyl, and the like. The alkyl group has 1 to 10 carbon atoms, preferably from 1 to 6 carbon atoms, more preferably methyl, ethyl propyl or isopropyl. The alkyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “aryl” is taken to include phenyl and naphthyl and may be optionally substituted by one or more C₁-C₄-alkyl, hydroxy, C₁-C₄-alkoxy, carbonyl, C₁-C₄-alkoxycarbonyl, C₁-C₄-alkylcarbonyloxy or halo.

The term “halo” is taken to include fluoro, chloro, bromo and iodo, preferably fluoro and chloro, more preferably fluoro. Reference to for example “haloalkyl” will include monohalogenated, dihalogenated and up to perhalogenated alkyl groups. Preferred haloalkyl groups are trifluoromethyl and pentafluoroethyl.

As used herein the term “chemotherapeutic agent” refers to any chemical substance having cytotoxic or anti-neoplastic activity, being capable of use in the treatment of disease (most typically cancer) and which has undesirable neurotoxic side effects associated with its administration. The neurotoxic side effects may be slight, moderate or severe in terms of the extent of neural dysfunction and/or damage caused or in terms of the symptoms experienced by the subject to which the agent is administered: Typically the neurotoxic side effects include restriction or degeneration of neurite outgrowth and/or one or more symptoms of neuropathy or a neuropathy-related condition.

Chemotherapeutic agents are commonly grouped according to their mode of action and/or the cellular target upon which they act. For example, chemotherapeutic agents may categorised as DNA-interactive agents (including topoisomerase inhibitors, DNA strand breakage agents and DNA minor groove binders), alkylating agents, antimetabolites, tubulin-interactive agents and hormonal agents.

Chemotherapeutic agents to which methods of the present application are applicable may be selected from any of these exemplary groups, but are not limited thereto. For a detailed discussion of chemotherapeutic agents and their method of administration, see Dorr, et al, Cancer Chemotherapy Handbook, 2d edition, pages 15-34, Appleton and Lang (Connecticut, 1994) herein incorporated by reference.

By way of example only, according to methods of the invention, chemotherapeutic agents may be selected from cisplatin, carboplatin, oxaliplatin, cyclophosphamide, altretamine, plicamydin, chlorambucil, chlormethine, Ifosfamide, melphalan, carmustine, fotemustine, lomustine, streptozocin, busulfan, dacarbazine, mechlorethamine, procarbazine, temozolomide, thioTEPA, uramustine, paclitaxel, docataxel, vinblastine, vincristine, vindesine, vinorelbine, hexamethylmelamine, etoposide, teniposide, methotrexate, pemetrexed, raltitrexed, cladribine, clofarabine, fludarabine, mercaptopurine, tioguanine, capecitabine, cytarabine, fluorouracil, fluxuridine, gemcitabine, daunorubicin, doxorubicin, epirubicin, idarubicin, mitoxantrone, valrubicin, bleomycin, hydroxyurea, mitomycin, topotecan, irinotecan, aminolevulinic acid, methyl aminolevulinate, porfimer sodium, verteporfin, alitretinoin, altretamine, amsacrine, anagrelide, arsenic trioxide, asparaginase, bexarotene, bortezomib, celecoxib, denileukin, diftitox, erlotinib, estramustine, gefitinib, hydroxycarbamide, imatinib, pentostatin, masoprocol, mitotane, pegaspargase, and tretinoin.

Phenoxodiol (2H-1-benzopyran-7-0,1,3-[4-hydroxyphenyl]), also known as dehydroequol, is an isoflavone analogue derived from genistein, which shows greater bio-availability and increased potency than its parent compound. Phenoxodiol and related compounds have been shown to have potent cytotoxic effects on a range of cancer cells, and to have protective ability against UV-induced immunosuppression and skin damage (see WO 98/08503 and WO 99/36050, the disclosures of which are incorporated herein by reference in their entirety). Additionally, phenoxodiol and related compounds have been demonstrated to sensitise tumours that are resistant to chemotherapeutic agents, thereby increasing the responsiveness of the tumours to a range of chemotherapeutic agents (WO 2004/030662, the disclosure of which is incorporated herein by reference in its entirety).

As disclosed herein the inventors have now surprisingly demonstrated that phenoxodiol acts as a neuroprotective agent against neuropathy or nerve damage that is induced by, or associated with, chemotherapy, thereby expanding further the clinical effects of this compound in treatments of cancer patients. As exemplified herein, phenoxodiol at low doses was found to be able to block neurite toxicity induced by at least cisplatin in the PC12 neuronal cell model. The sensitivity of neurite toxicity to the protective effect of phenoxodiol was approximately 10 fold higher than the cytotoxic and anti-proliferative effects observed in a variety of cancer cell lines. Significant protective effects of phenoxodiol on neurite toxicity were observed at 1 μM phenoxodiol, which is within the concentration range of phenoxodiol for which cytotoxic and anti-proliferative activity has previously been observed in a variety of cells. However notably, as disclosed herein the inventors have found that significant neurite protective effects of phenoxodiol are observed at a 10 fold lower concentration.

The neuroprotective effects exhibited by phenoxodiol may therefore facilitate the continued employment of chemotherapeutic treatment regimes where such regimes would otherwise have been ceased, or at least the dosage of the chemotherpaeutic agent reduced.

One aspect of the present invention provides a method for treating or preventing neuropathy or a neuropathy-related condition in a subject, wherein the neuropathy or neuropathy-related condition is induced by, or otherwise associated with, at least one chemotherapeutic agent, the method comprising administering to the subject an effective amount of an isoflavonoid compound of formula (I):

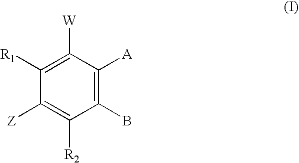

in which
R₁, R₂ and Z are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₂ is as previously defined, and R₁ and Z taken together with the carbon atoms to which they are attached form a five-membered ring selected from

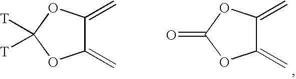

or
R₁ is as previously defined, and R₂ and Z taken together with the carbon atoms to which they are attached form a five-membered ring selected from

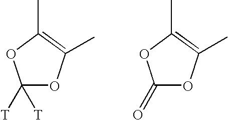

and
W is R₁, A is hydrogen, hydroxy, NR₃R₄ or thio, and B is selected from

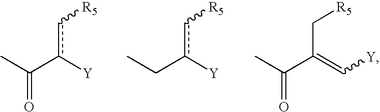

or
W is R₁, and A and B taken together with the carbon atoms to which they are attached form a six-membered ring selected from

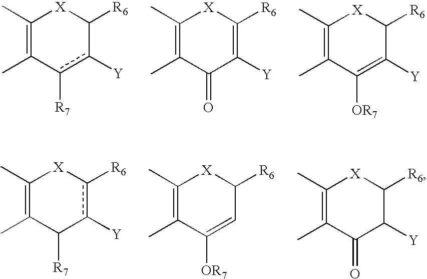

or
W, A and B taken together with the groups to which they are associated are selected from

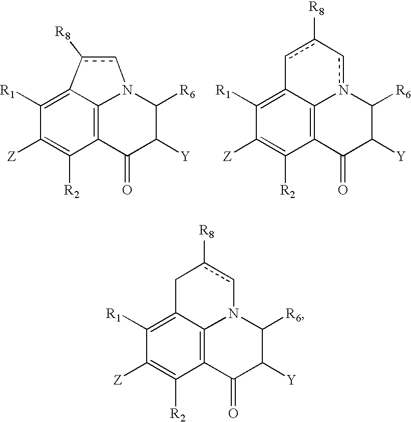

or
W and A taken together with the groups to which they are associated are selected from

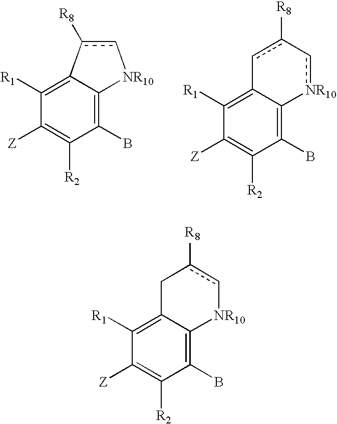

and B is selected from

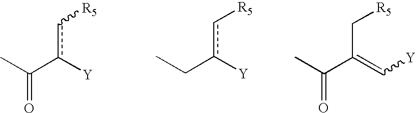

wherein
R₃ is hydrogen, alkyl, arylalkyl, alkenyl, aryl, an amino acid, C(O)R₁₁ where R₁₁ is hydrogen, alkyl, aryl, arylalkyl or an amino acid, or CO₂R₁₂ where R₁₂ is hydrogen, alkyl, haloalkyl, aryl or arylalkyl,
R₄ is hydrogen, alkyl or aryl, or
R₃ and R₄ taken together with the nitrogen to which they are attached comprise pyrrolidinyl or piperidinyl,
R₅ is hydrogen, C(O)R₁₁ where R₁₁ is as previously defined, or CO₂R₁₂ where R₁₂ is as previously defined,
R₆ is hydrogen, hydroxy, alkyl, aryl, amino, thio, NR₃R₄, COR₁₁ where R₁₁ is as previously defined, CO₂R₁₂ where R₁₂ is as previously defined or CONR₃R₄,
R₇ is hydrogen, C(O)R₁₁ where R₁₁ is as previously defined, alkyl, haloalkyl, alkenyl, aryl, arylalkyl or Si(R₁₃)₃ where each R₁₃ is independently hydrogen, alkyl or aryl,
R₈ is hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀ is hydrogen, alkyl, haloalkyl, amino, aryl, arylalkyl, an amino acid, alkylamino or dialkylamino, the drawing [symbol] represents either a single bond or a double bond,
T is independently hydrogen, alkyl or aryl,
X is O, NR₄ or S, and
Y is

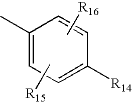

wherein

R₁₄, R₁₅ and R₁₀ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, thio, alkylthio, amino, alkylamino, dialkylamino, nitro or halo, or any two of R₁₄, R₁₅ and R₁₆ are fused together to form a cyclic alkyl, aromatic or heteroaromatic structure, and pharmaceutically acceptable salts thereof.

According to methods of the invention, isoflavonoid compounds of formula (I) may be selected from general formulae (III)-(IX), typically from general formulae (IV)-(IX):

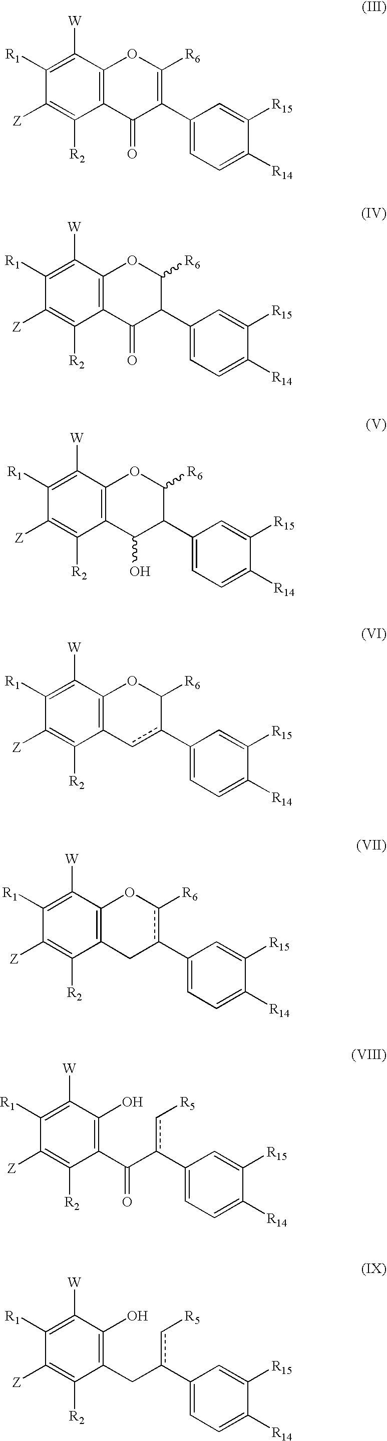

in which
R₁, R₂, R₅, R₆, R₁₄, R₁₅, W and Z are as defined above,
more typically
R₁, R₂, R₁₄, R₁₅, W and Z are independently hydrogen, hydroxy, OR₉, OC(O)R₁₀, C(O)R₁₀, COOH, CO₂R₁₀, alkyl, haloalkyl, arylalkyl, aryl, thio, alkylthio, amino, alkylamino, dialkylamino, nitro or halo,
R₅ is hydrogen, C(O)R₁₁ where R₁₁ is hydrogen, alkyl, aryl, or an amino acid, or CO₂R₁₂ where R₁₂ is hydrogen, alkyl or aryl,
R₆ is hydrogen, hydroxy, alkyl, aryl, COR₁₁ where R₁₁ is as previously defined, or CO₂R₁₂ where R₁₂ is as previously defined,
R₉ is alkyl, haloalkyl, arylalkyl, or C(O)R₁₁ where R₁₁ is as previously defined, and
R₁₀ is hydrogen, alkyl, amino, aryl, an amino acid, alkylamino or dialkylamino,
more typically
R₁ and R₁₄ are independently hydroxy, OR₉, OC(O)R₁₀ or halo,
R₂, R₁₅, W and Z are independently hydrogen, hydroxy, OR₉, OC(O)R₁₀, C(O)R₁₀, COOH, CO₂R₁₀, alkyl, haloalkyl, or halo,

R₅ is hydrogen, C(O)R₁₁ where R₁₁ is hydrogen or alkyl, or CO₂R₁₂ where R₁₂ is hydrogen or alkyl,
R₆ is hydrogen or hydroxy,
R₉ is alkyl, arylalkyl or C(O)R₁₁ where R₁₁ is as previously defined, and
R₁₀ is hydrogen or alkyl,
and more typically
R₁ and R₁₄ are independently hydroxy, methoxy, benzyloxy, acetyloxy or chloro,
R₂, R₁₅, W and Z are independently hydrogen, hydroxy, methoxy, benzyloxy, acetyloxy, methyl, trifluoromethyl or chloro,
R₅ is hydrogen or CO₂R₁₂ where R₁₂ is hydrogen or methyl, and
R₆ is hydrogen.

In particular embodiments, isoflavonoid compounds of formula (I) are selected from:

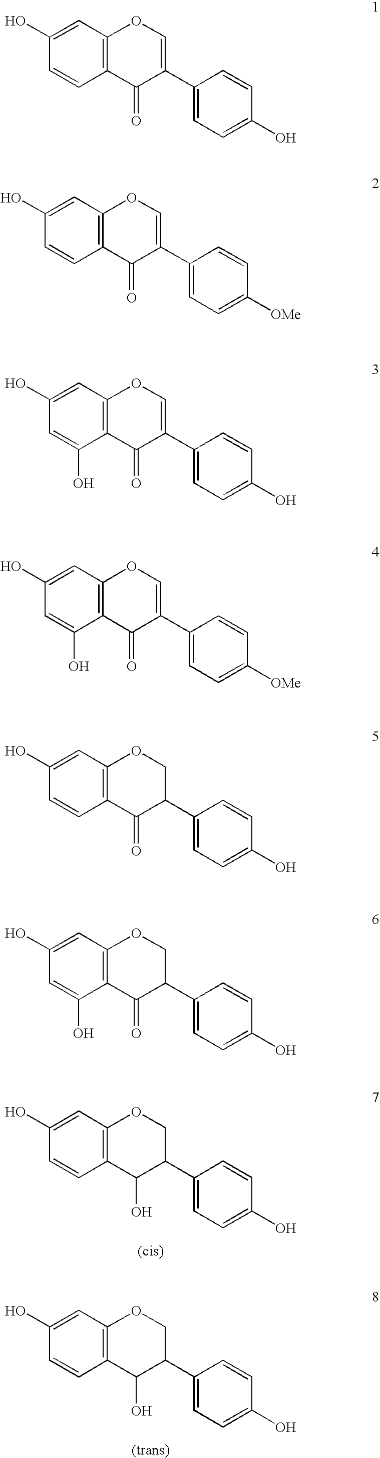

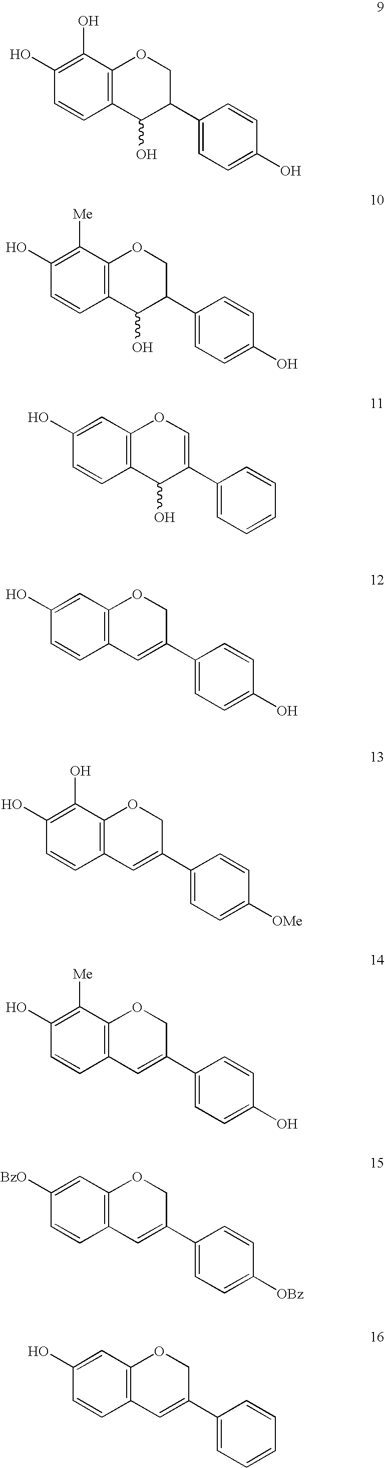

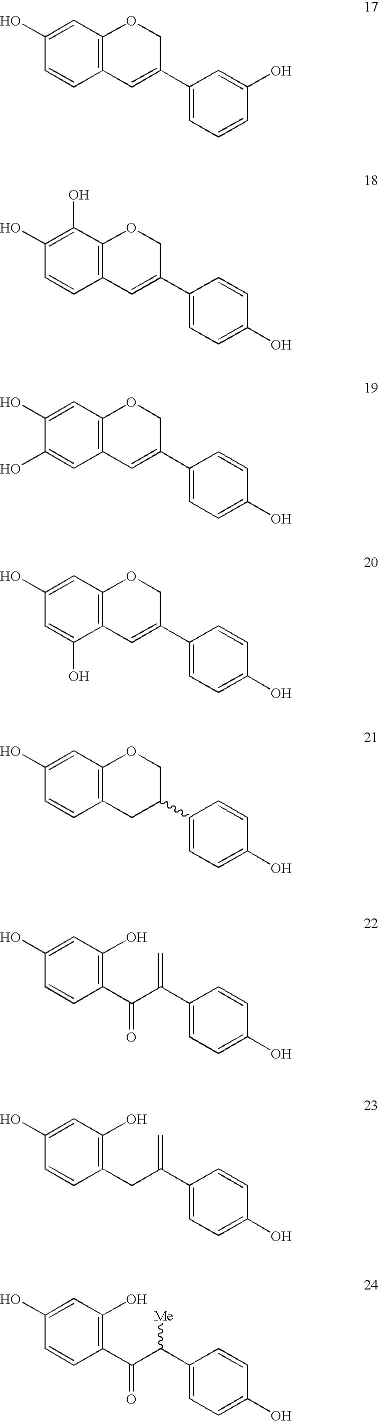

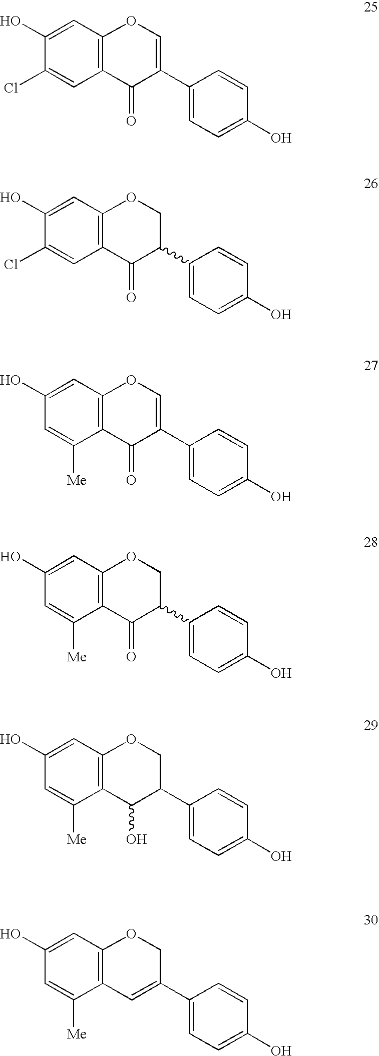

In further embodiments the isoflavonoid compounds are the isoflav-3-ene and isoflavan compounds of general formula (VI), and the 3-ene compounds of the general formula (VIa):

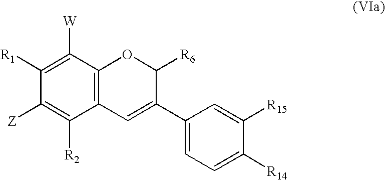

in which
R₁, R₂, R₆, R₁₄, R₁₅, W and Z are as defined above;
more typically
R₁, R₂, R₁₄, R₁₅, W and Z are independently hydrogen, hydroxy, OR₉, OC(O)R₁₀, C(O)R₁₀, COOH, CO₂R₁₀, alkyl, haloalkyl, arylalkyl, aryl, thio, alkylthio, amino, alkylamino, dialkylamino, nitro or halo,
R₆ is hydrogen, hydroxy, alkyl, aryl, COR₁₁ where R₁₁ is as previously defined, or CO₂R₁₂ where
R₁₂ is as previously defined,
R₉ is alkyl, haloalkyl, arylalkyl, or, C(O)R₁₁ where R₁₁ is as previously defined, and R₁₀ is hydrogen, alkyl, amino, aryl, an amino acid, alkylamino or dialkylamino,
more typically
R₁ is hydroxy, OR₉, OC(O)R₁₀ or halo,
R₂, R₁₄, R₁₅, W and Z are independently hydrogen, hydroxy, OR₉, OC(O)R₁₀, C(O)R₁₀, COOH, CO₂R₁₀, alkyl, haloalkyl, or halo,
R₆ is hydrogen,
R₉ is alkyl, arylalkyl or C(O)R₁₁ where R₁₁ is as previously defined, and R₁₀ is hydrogen or alkyl,
and more typically
R₁ is hydroxy, methoxy, benzyloxy, acetyloxy or chloro,
R₂, R₁₄, R₁₅, W and Z are independently hydrogen, hydroxy, methoxy, benzyloxy, acetyloxy, methyl, trifluoromethyl or chloro, and
R₆ is hydrogen,
   including pharmaceutically acceptable salts and derivatives thereof.

In a particular embodiment of the invention the isoflavonoid compound is phenoxodiol, also known as dehydroequol (compound 12 as defined above).

According to the methods of present invention isoflavonoid compounds and compositions comprising such isoflavonoids may be administered by any suitable route, either systemically, regionally or locally. The particular route of administration to be used in any given circumstance will depend on a number of factors, including the nature of the condition to be treated, the severity and extent of the condition, the required dosage of the particular compound to be delivered and the potential side-effects of the compound. Additionally, in particular embodiments it may be advantageous to administer the isoflavonoid via the same route as the chemotherapeutic agent. This may enable their concurrent administration or inclusion of both agents into a single pharmaceutical composition.

For example, in circumstances where it is required that appropriate concentrations of the desired compound are delivered directly to the site in the body to be treated, administration may be regional rather than systemic. Regional administration provides the capability of delivering very high local concentrations of the desired compound to the required site and thus is suitable for achieving the desired therapeutic or preventative effect whilst avoiding exposure of other organs of the body to the compound and thereby potentially reducing side effects.

By way of example, administration according to embodiments of the invention may be achieved by any standard routes, including intracavitary, intravesical, intramuscular, intraarterial, intravenous, intraocular, subcutaneous, topical or oral.

In employing methods of the invention, isoflavonoid compounds may be formulated in pharmaceutical compositions. Suitable compositions may be prepared according to methods which are known to those of ordinary skill in the art and may include a pharmaceutically acceptable diluent, adjuvant and/or excipient. The diluents, adjuvants and excipients must be “acceptable” in terms of being compatible with the other ingredients of the composition, and not deleterious to the recipient thereof. The diluent, adjuvant or excipient may be a solid or a liquid, or both, and may be formulated with the compound as a unit-dose, for example, a tablet, which may contain from 0.5% to 59% by weight of the active compound, or up to 100% by weight of the active compound. One or more active compounds may be incorporated in the formulations of the invention, which may be prepared by any of the well known techniques of pharmacy consisting essentially of admixing the components, optionally including one or more accessory ingredients.

Examples of pharmaceutically acceptable diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 1% to 99.9% by weight of the compositions.

Formulations suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more accessory ingredients. Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Formulations suitable for buccal (sublingual) administration include lozenges comprising the active compound in a flavoured base, usually sucrose and acacia or tragacanth; and pastilles comprising the compound in an inert base such as gelatin and glycerin or sucrose and acacia.

Compositions of the present invention suitable for parenteral administration typically conveniently comprise sterile aqueous preparations of the active compounds, which preparations may be isotonic with the blood of the intended recipient. These preparations are typically administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound(s) and are administered at a rate of 0.1 ml/minute/kg or as appropriate. Parenteral administration is a preferred route of administration for the compounds of the present invention.

Formulations suitable for rectal administration are typically presented as unit dose suppositories. These may be prepared by admixing the active compound with one or more conventional solid carriers, for example, cocoa butter, and then shaping the resulting mixture.

Formulations or compositions suitable for topical administration to the skin may take the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 0.5% w/w, for example, from 0.5% to 2% w/w. Examples of such compositions include cosmetic skin creams.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound. Formulations suitable for transdermal administration may also be delivered by iontophoresis (see, for example, Pharmaceutical Research 3 (6), 318 (1986)) and typically take the form of an optionally buffered aqueous solution of the active compound. For example, suitable formulations may comprise citrate or bis/tris buffer (pH 6) or ethanol/water and contain from 0.1 M to 0.2 M active ingredient.

The active compounds may be provided in the form of food stuffs, such as being added to, admixed into, coated, combined or otherwise added to a food stuff. The term food stuff is used in its widest possible sense and includes liquid formulations such as drinks including dairy products and other foods, such as health bars, desserts, etc. Food formulations containing compounds of the invention can be readily prepared according to standard practices.

According to the present invention, compounds and compositions may be administered either therapeutically or preventively. In a therapeutic application, compounds and compositions are administered to a patient already suffering from a condition or experiencing symptoms, in an amount sufficient to cure or at least partially arrest the condition, symptoms and/or any associated complications. The compound or composition should provide a quantity of the active compound sufficient to effectively treat the patient.

The effective dose level of the administered compound for any particular subject will depend upon a variety of factors including: the type of condition being treated and the stage of the condition; the activity of the compound employed; the composition employed; the age, body weight, general health, sex and diet of the patient; the time of administration; the route of administration; the rate of sequestration of compounds; the duration of the treatment; drugs used in combination or coincidental with the treatment, together with other related factors well known in medicine.

One skilled in the art would be able, by routine experimentation, to determine an effective, non-toxic dosage which would be required to treat applicable conditions. These will most often be determined on a case-by-case basis. By way of example only, an effective dosage may be expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; or about 1.0 mg to about 250 mg per kg body weight per 24 hours. More typically, an effective dose range is expected to be in the range of about 10 mg to about 200 mg per kg body weight per 24 hours.

Further, it will be apparent to those of ordinary skill in the art that the optimal quantity and spacing of individual dosages will principally be determined by the nature and extent of the condition being treated, the form, route and site of administration, and the individual being treated. Suitable conditions can be determined by conventional techniques.

It will also be apparent to those of ordinary skill in the art that the optimal course of treatment, such as, the number of doses of the composition given per day for a defined number of days, can be ascertained by those skilled in the art using conventional course of treatment determination tests.

In accordance with the methods of the invention, isoflavonoid compounds or pharmaceutically acceptable derivatives prodrugs or salts thereof can be co-administered with other active materials that do not impair the desired action, or with materials that supplement the desired action, such as antibiotics, antifungals, antiinflammatories, lipid lowering agents, platelet aggregation inhibitors, antithrombotic agents, calcium channel blockers, corticosteroids or antiviral compounds.

The co-administration of agents may be simultaneous or sequential. Simultaneous administration may be effected by the compounds being formulated in a single composition, or in separate compositions administered at the same or similar time. Sequential administration may be in any order as required.

The isoflavonoids of formula (I) for use in the present invention may be derived from any number of sources readily identifiable to a person skilled in the art. They may be obtained in the form of concentrates or extracts from plant sources. Again, those skilled in the art will readily be able to identify suitable plant species, however, for example, plants of particular use in the invention include leguminous plants. More preferably, an extract comprising isoflavonoids is obtained from soy, chickpea, lentils, beans, red clover or subterranean clover species and the like. Suitable methods for the extraction of such extracts are described, for example, in International Patent Application published under WO 98/49153 (the disclosure of which is incorporated herein in its entirety by reference).

Alternatively isoflavonoids for use in accordance with the invention may be derived synthetically. For example International Patent Applications published under WO 98/08503 and WO 00/49009 (the disclosures of which are incorporated herein in their entirety by reference) and references cited therein provide general synthetic methods for the preparation of isoflavonoid compounds for use in the present invention.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

### Example 1

### Neurite Toxicity in the Presence of Cisplatin and Paclitaxel

Optimal treatment concentrations of cisplatin and paclitaxel that caused neurite damage were determined by treating differentiated PC12 cells for 24 hours with serial dilutions of either drug.

PC-12 cells were maintained in Dulbecco's Modified Eagle Media (DMEM; Gibco) with 10% calf serum (Turbo calf serum, Invitrogen), 5% horse serum (JRH Biosciences, Victoria, Australia) and 1% penicillin/streptomycin (Invitrogen) at 37° C., in a 5% CO₂ atmosphere. Differentiation into neurons was achieved by seeding cells at a density of 15,000 cells/well in 24-well plates (Falcon Becton Dickinson), on 13 mm glass cover slips (Menzel Glaser, Germany) coated with laminin (Invitrogen) and poly-DL-ornithine (Sigma) in DMEM plus 1% horse serum and 50 ng/mL of nerve growth factor (NGF) (Sigma). The cells were incubated for 72 hours under differentiation conditions, before use in the neurite outgrowth assays below.

Cisplatin (Sigma) and paclitaxel (Sigma) were diluted in DMSO and stocks maintained at −20° C. Working concentrations were diluted in PC12 cell differentiation medium as above. Treatments consisted of a 24 hour incubation in differentiation media with various concentrations of either agent. Working concentrations of cisplatin ranged from 0.5 μg/ml to 100 μg/ml, and working concentrations of paclitaxel ranged from 0.156 μg/ml and 10 μg/ml.

Outgrowing projections were considered neurites if they were greater than a cell body width (10 μm), essentially as previously described. Images of all neurons in thirty randomly selected fields were digitally captured using an Olympus BX60 fluorescence microscope with a UPIanFI 40×/0.75 objective lens and a Zeiss Axiocam HRc digital camera and Zeiss Axiovision 3.1 software. Neurons in all fields were counted per condition and the number of cells with neurites expressed as a percentage of total cells present and error expressed as the standard error of the mean (sem). Each experiment was conducted in triplicate and the results pooled. Statistical significance of data was analysed using ANOVA followed by the Bonferroni post-hoc test.

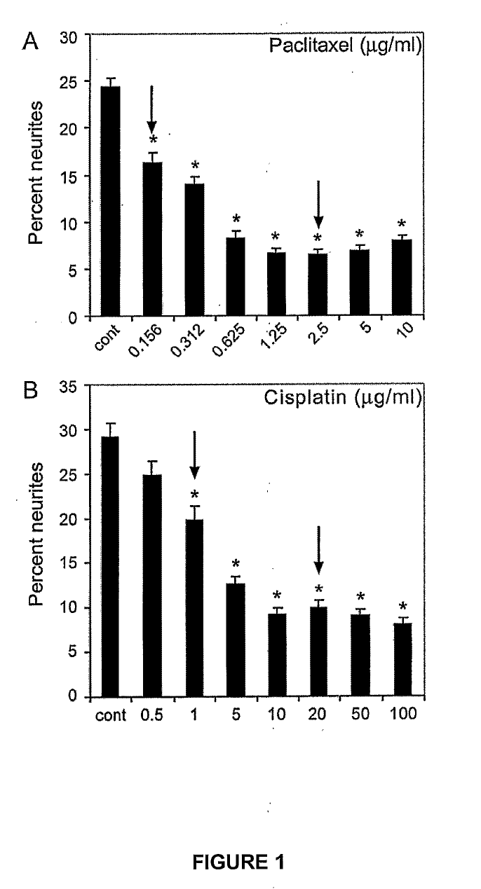

The concentration which caused the greatest reduction in the percentage of cells with neurites, without killing the cells, was chosen as a strong dose for subsequent experiments. A moderate dose was also selected, where the drug reduced neurite outgrowth by approximately 50% of the strong dose. Paclitaxel caused a 73.6% reduction in the percentage of cells with neurites at a concentration of 2.5 μg/mL (2.93 μM). This was designated as the strong dose, where as the selected moderate dose, at 0.156 μg/mL (0.18 μM) produced a 33% decrease in the percentage of cells possessing neurites (FIG. 1A). The strong dose of cisplatin was selected as 20 μg/mL (66.65 μM), which reduced the percentage of cells with neurites by 65.8%, and the moderate dose of 1 μg/mL (3.33 μM) caused a 31.9% reduction in percentage of cells with neurites (FIG. 1B). The chosen concentrations did not produce cytotoxicity (data not shown). It should be noted that NGF blocks cisplatin induced cytotoxicity of PC12 cells. Hence, in the present study in which the PC12 cells were maintained in a differentiated state in the presence of NGF, the effects of cisplatin on neurite toxicity were able to be clearly isolated from that of cytotoxicity.

A serial dilution was also conducted for phenoxodiol to determine the concentration which would not affect normal growth of the differentiated cells. As for cisplatin and paclitaxel, phenoxodiol was diluted in DMSO and stocks maintained at −20° C. Working concentrations were diluted in PC12 cell differentiation medium as above.

In addition to the maximal concentration tested that did not affect survival of PC12 cells (1 μM; 3.2 μg/ml), two other concentrations were selected for treatments, one log above (10 μM; 32 μg/ml) and one log below (100 nM; 320 ng/ml). Doses up to 1 μM had no effect on cell death or neurite outgrowth (data not shown), although a 10 μM concentration showed considerable cytotoxicity (data not shown).

### Example 2

### Effect of Phenoxodiol in Blocking Cisplatin and Paclitaxel Neurite Toxicity

To determine whether phenoxodiol could block cisplatin or paclitaxel induced neurite toxicity, three different concentrations of phenoxodiol were added to cells in combination with cisplatin or paclitaxel. Combination treatments consisted of a 24 hour incubation in differentiation media with various combinations of strong or moderate doses (so as to produce strong or moderate neurite toxicitiy) of cisplatin (20 μg/ml/66.65 μM and 1 μg/ml/3.33 μM) and paclitaxel (2.4 μg/ml/2.93 μM and 0.156 μg/ml/0.18 μM) and three doses of phenoxodiol (100 nM, 1 μM, 10 μM) as described in Example 1. Neurite toxicity was determined as the percent of cells comprising neurite outgrowths as described in Example 1.

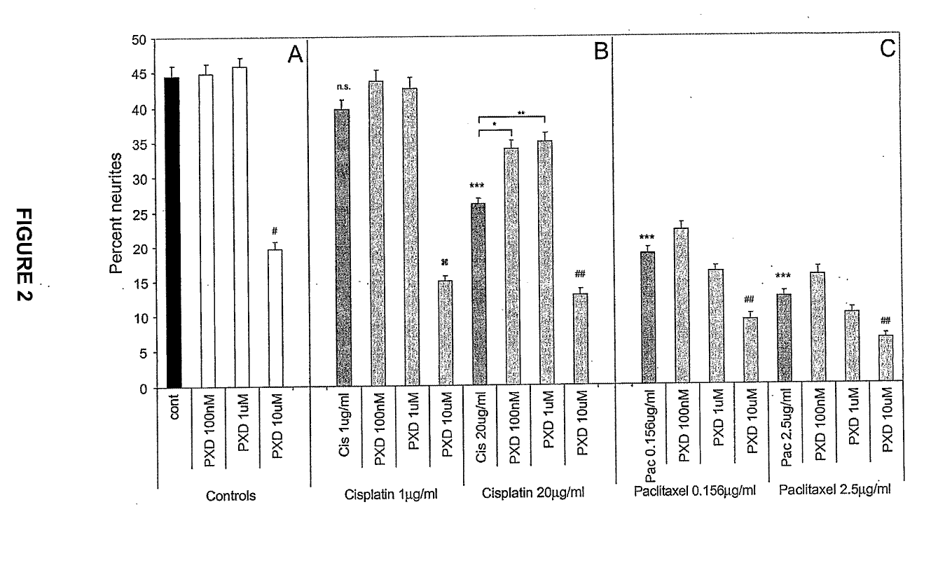

Phenoxodiol had no effect on percent neurites at 100 nM or 1 μM but showed significant neurite toxicity at 10 μM (# P<0.001 compared to no treatment control) (FIG. 2A). This neurite toxicity was exacerbated in combination with cisplatin and paclitaxel, with increased toxicity compared to phenoxodiol at 10 μM alone (P<0.01 in combination with cisplatin 1 μg/ml; P<0.001 in combination with cisplatin 20 μg/ml or both concentrations of paclitaxel) (FIGS. 2B, C).

Cisplatin alone at 1 μg/ml showed a non-significant (n.s.) trend for moderate toxicity when assessed by ANOVA, although a direct comparison of control versus cisplatin at 1 μg/ml using the t-test was significant (P<0.02) (FIG. 2B). Comparison of cisplatin 1 μg/ml with cisplatin 1 μg/ml+phenoxodiol 1 μM demonstrated that protection of this cisplatin induced neurite toxicity was significant by t-test (P<0.02). Robust neurite toxicity was observed with cisplatin alone at 20 μg/ml, with a 42% decrease in percent neurites compared to control (*** P<0.001) (FIG. 2B). This strong neurite toxicity was blocked by phenoxodiol at 100 nM (p<0.01) and 1 μM (p<0.001).

Paclitaxel produced robust neurite toxicity at both doses tested. The moderate dose caused a 58% reduction in cells with neurites (*** p<0.001) and the strong dose produced a 72% reduction (*** p<0.001) compared to no treatment control (FIG. 2C). When combined with phenoxodiol at 100 nM, there was an increase in cells with neurites, although this was not determined to be statistically significant.

To determine whether there were more subtle effects on neurite toxicity than could be measured by counting the percent of cells with neurite as above, the effect of Cisplatin, Paclitaxel and phenoxodiol on neurite length was examined. Further to the above described analysis of cells containing neurites, the longest neurite per neuron present in each frame was measured using Image J open source software (NIH, USA).

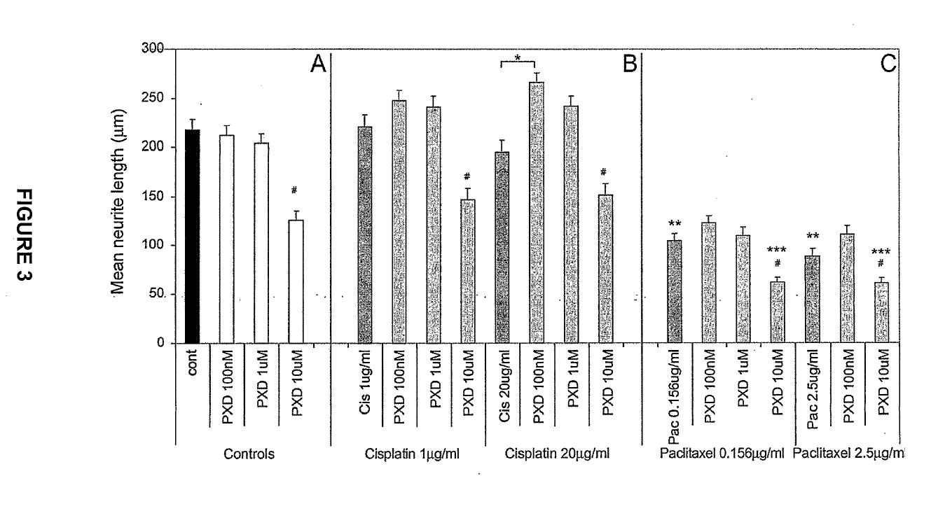

The 10 μM concentration of phenoxodiol resulted in significant reductions in average neurite length compared to no treatment control, both alone (FIG. 3A) and in combination with Cisplatin and Paclitaxel (FIG. 3b, c) (# p<0.001). While cisplatin decreased the percentage of cells that had neurites (FIGS. 1, 2), it had no significant effect on the average neurite length of the remaining neurites (FIG. 3B). Interestingly however, while phenoxodiol (100 nM and 1 μM) or cisplatin alone had no effect on neurite length, the combination of these drugs increased neurite length compared to cisplatin alone (* p<0.001), reflecting a blocking of cisplatin-induced neurite toxicity. Paclitaxel reduced neurite length compared to the no treatment control, at both concentrations used (** p<0.001). This inhibition appeared to be partially ameliorated by addition of phenoxodiol at 100 nM or 1 μM as determined by a slight increase in neurite length (FIG. 3C).

Further, immunocytochemistry was used to visualise the neurite toxicity effects described above. To do so, following the treatment period, cells were washed with PBS, fixed with 4% paraformaldehyde and permeabilised with ice-cold methanol. Neurons were immunostained for the neuronal marker βIII-tubulin (Promega; Madison, Wisconson, USA) and a Cy3-conjugated anti-mouse antibody (Invitrogen) was used to visualise the staining.

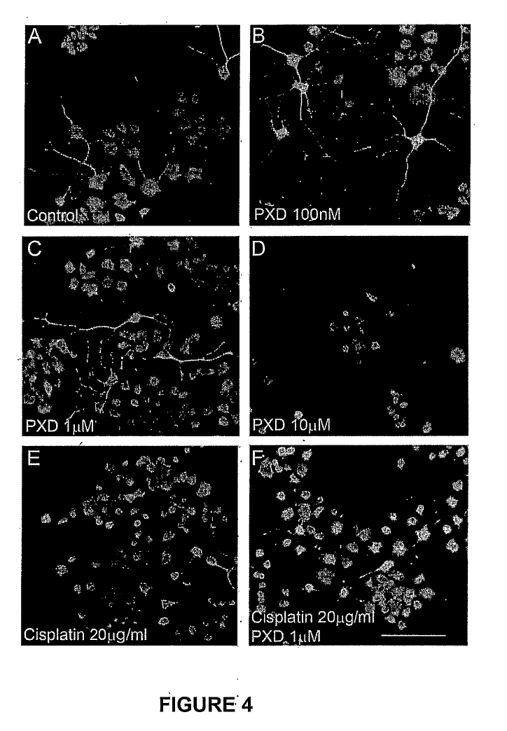

Control cultures (FIG. 4A) showed a large percentage of cells with long neurites, which were also present in 100 nM and 1 μm phenoxodiol (FIG. 4B, C). Significant cell and neurite toxicity was observed in cells incubated with 10 μm phenoxodiol (FIG. 4D). Cisplatin at 20 μg/ml decreased the percentage of cells bearing neurites compared to control (FIG. 4E), which was blocked by 1 μm phenoxodiol (FIG. 4F).

### Example 3

### Effect of Phenoxodiol in Enhancing Recovery from Cisplatin- and Paclitaxel-Induced Neurite Toxicity

To determine whether phenoxodiol could have an effect on recovery from cisplatin- or paclitaxel-induced neurite toxicity by exacerbation, reversal, retardation or facilitation of repair, recovery experiments were performed. When conducting the recovery experiments, the concentrations of phenoxodiol tested were decreased by 1 log to 10 nM 100 nM and 1 μM. Differentiated PC12 cells were incubated with Cisplatin or Paclitaxel for 24 hrs as described in Example 2, then washed off and cells allowed to recover for 24 hrs in fresh differentiation media before phenoxodiol was added for a further 24 hrs.

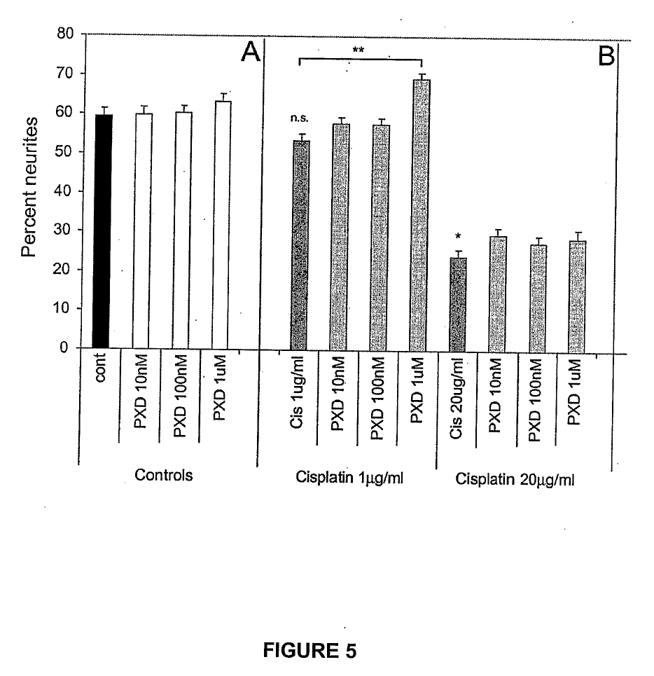

Addition of phenoxodiol at 10 nM, 100 nM or 1 μM alone had no effect on neurite outgrowth (FIG. 5A). After 48 hrs of recovery, cisplatin showed no neurite toxicity at 1 μg/ml but greater than 50% toxicity at 20 μg/ml (*p<0.001) compared to the no treatment control (FIG. 5B), indicating that the cells could not recover from this higher dose by 48 hrs after drug addition. Phenoxodiol showed a slight effect on recovery of neurite outgrowth following 20 μg/ml Cisplatin treatment, whilst there was a more significant enhancement of neurite outgrowth by phenoxodiol (1 μM), following Cisplatin at 1 μg/ml (** p<0.001) (FIG. 5B).

### REFERENCES

Gill J S, Windebank A J. Cisplatin-induced apoptosis in rat dorsal root ganglion neurons is associated with attempted entry into the cell cycle. J Clin Invest 1998; 101(12):2842-50.
Goldschmit Y, Walters C E, Scott H J, Greenhalgh C J, Turnley A M. SOCS2 induces neurite outgrowth by regulation of epidermal growth factor receptor activation. J Biol Chem 2004; 279(16):16349-55.
Letourneau P C, Ressler A H. Inhibition of neurite initiation and growth by taxol. J Cell Biol 1984; 98(4): 1355-62.
Macdonald, D R. Neurologic complications of chemotherapy. Neurol Olin 1991; 9(4):955/967.
McDonald E S, Randon K R, Knight A, Windebank A J. Cisplatin preferentially binds to DNA in dorsal root ganglion neurons in vitro and in vivo: a potential mechanism for neurotoxicity. Neurobiol Dis 2005; 18(2):305-13.
Quasthoff, S and Hartung, H P. Chemotherapy-induced peripheral neuropathy. J Neurol 2002; 249(1):9-17.
Scott H J, Stebbing M J, Walters C E, McLenachan S, Ransome M I, Nichols N R, et al. Differential effects of SOCS2 on neuronal differentiation and morphology. Brain Res 2006; 1067(1):138-45.
Zwelling L A, Michaels S, Schwartz H, Dobson P P, Kohn K W. DNA cross-linking as an indicator of sensitivity and resistance of mouse L1210 leukemia to cisdiamminedichloroplatinum(II) and L-phenylalanine mustard. Cancer Res 1981; 41(2):640-9.

1. A method for treating or preventing neuropathy or a neuropathy-related condition in a subject, wherein the neuropathy or neuropathy-related condition is induced by, or otherwise associated with, treatment of the subject with at least one chemotherapeutic agent, the method i comprising administering to the subject an effective amount of an isoflavonoid compound of formula (I): 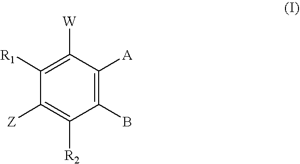
in which
R₁, R₂ and Z are independently hydrogen, hydroxy, OR₉ OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₂ is as previously defined, and R₁ and Z taken together with the carbon atoms to which they are attached form a five-membered ring selected from 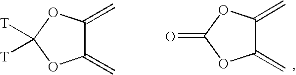
or
R₁ is as previously defined, and R₂ and Z taken together with the carbon atoms to which they are attached form a five-membered ring selected from 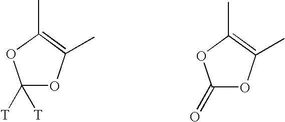
and
W is R₁, A is hydrogen, hydroxy, NR₃R₄ or thio, and B is selected from 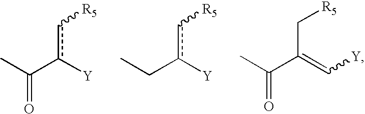
or
W is Rᵢ, and A and B taken together with the carbon atoms to which they are attached form a six-membered ring selected from 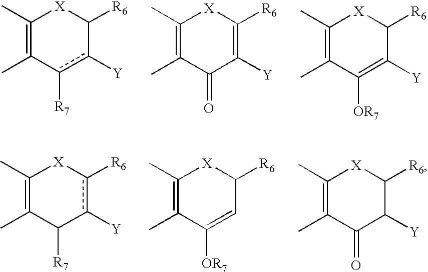
or
W, A and B taken together with the groups to which they are associated are selected from 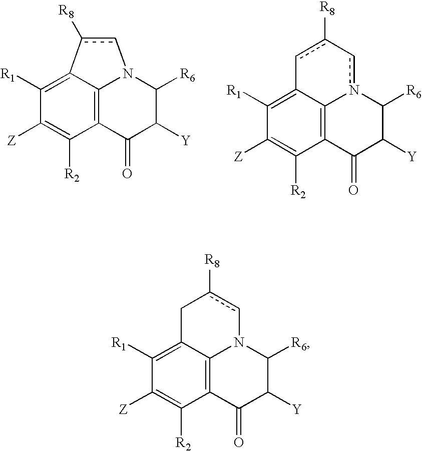
or
W and A taken together with the groups to which they are associated are selected from 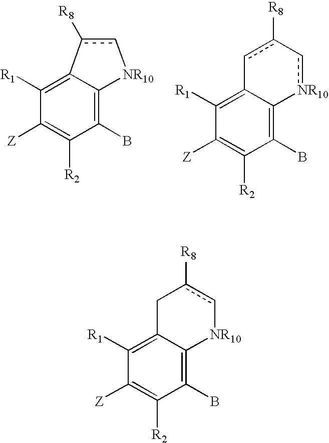
and B is selected from 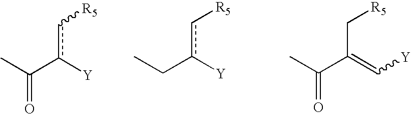
wherein
R₃ is hydrogen, alkyl, arylalkyl, alkenyl, aryl, an amino acid, C(O)R₁₁ where R₁₁ is hydrogen, alkyl, aryl, arylalkyl or an amino acid, or CO₂R₁₂ where R₁₂ is hydrogen, alkyl, haloalkyl, aryl or arylalkyl,
R₄ is hydrogen, alkyl or aryl, or
R₃ and R₄ taken together with the nitrogen to which they are attached comprise pyrrolidinyl or piperidinyl,
R₅ is hydrogen, C(O)R₁₁ where R₁₁ is as previously defined, or CO₂R₁₂ where R₁₂ is as previously defined,
R₆ is hydrogen, hydroxy, alkyl, aryl, amino, thio, NR₃R₄, COR₁₁ where R₁₁ is as previously defined, CO₂R₁₂ where R₁₂ is as previously defined or CONR₃R₄,
R₇ is hydrogen, C(O)R₁₁ where R₁₁ is as previously defined, alkyl, haloalkyl, alkenyl, aryl, arylalkyl or Si(R₁₃)₃ where each R₁₃ is independently hydrogen, alkyl or aryl,
R₈ is hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀ is hydrogen, alkyl, haloalkyl, amino, aryl, arylalkyl, an amino acid, alkylamino or dialkylamino, the drawing [symbol] represents either a single bond or a double bond, T is independently hydrogen, alkyl or aryl,
X is O, NR₄ or S, and Y is 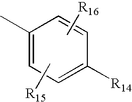
wherein
R₁₄, R₁₅ and R₁₆ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, thio, alkylthio, amino, alkylamino, dialkylamino, nitro or halo, or any two of R₁₄, R₁₅ and R₁₆ are fused together to form a cyclic alkyl, aromatic or heteroaromatic structure, and pharmaceutically acceptable salts thereof.

2. The method of claim 1 wherein the chemotherapeutic agent is any agent used in the treatment of cancer or tumours, and wherein administration of the agent causes nerve dysfunction and/or damage, typically peripheral nerves.

3. The method of claim 2 wherein the dysfunction and/or damage caused by the chemotherapeutic agent is of peripheral nerves.

4. The method of claim 1 wherein the chemotherapeutic agent is selected from the group consisting of: cisplatin, carboplatin, paclitaxel, docetaxel, vincristine, vinorelbine, hycamtin, hexamethylmelamine, bortezomib, cytarabine and procarbazine, and analogues or derivatives thereof.

5. The method of claim 4 wherein the chemotherapeutic agent is cisplatin or an analogue or derivative thereof.

6. The method of claim 1 wherein the isoflavonoid is administered prior to administration of the chemotherapeutic agent.

7. The method of claim 1 wherein the isoflavonoid is administered simultaneously or in conjunction with the chemotherapeutic agent.

8. The method of claim 1 wherein the isoflavonoid is administered following administration of the chemotherapeutic agent.

9. The method of claim 1 wherein the isoflavonoid and the chemotherapeutic agent are administered via the same route.

10. The method of claim 1 wherein the isoflavonoid and the chemotherapeutic agent are administered via different routes,

11. The method of claim 1 wherein the isoflavonoid is selected from the group consisting of:

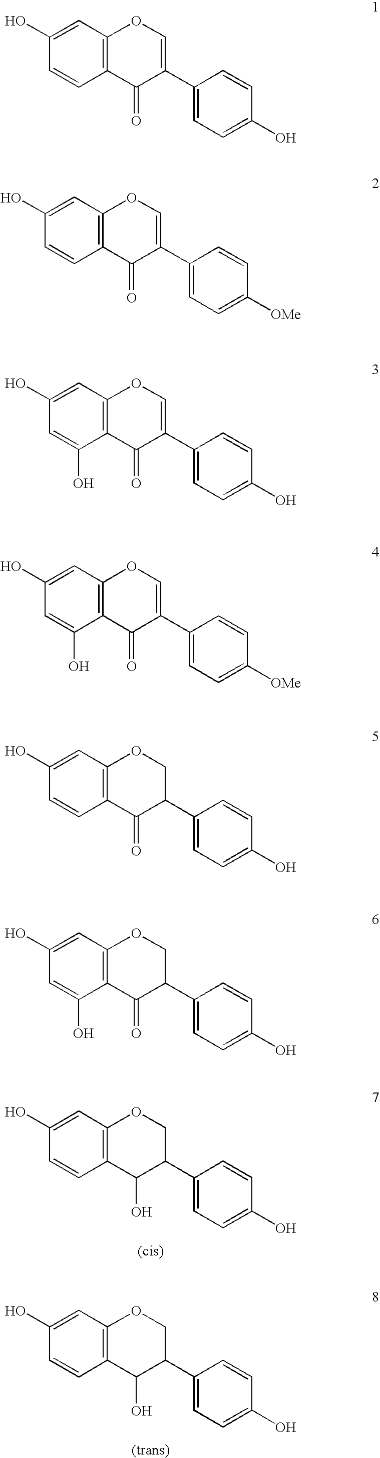

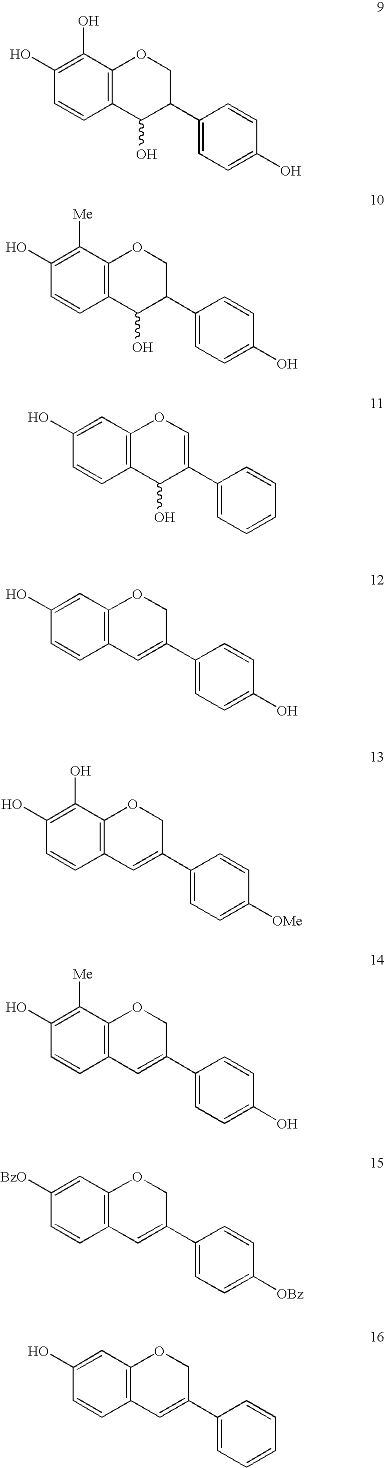

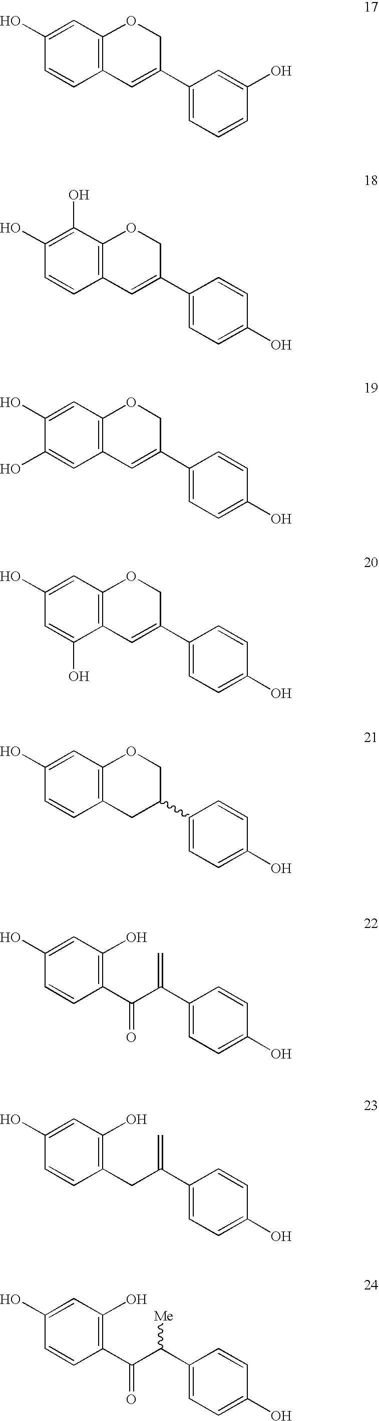

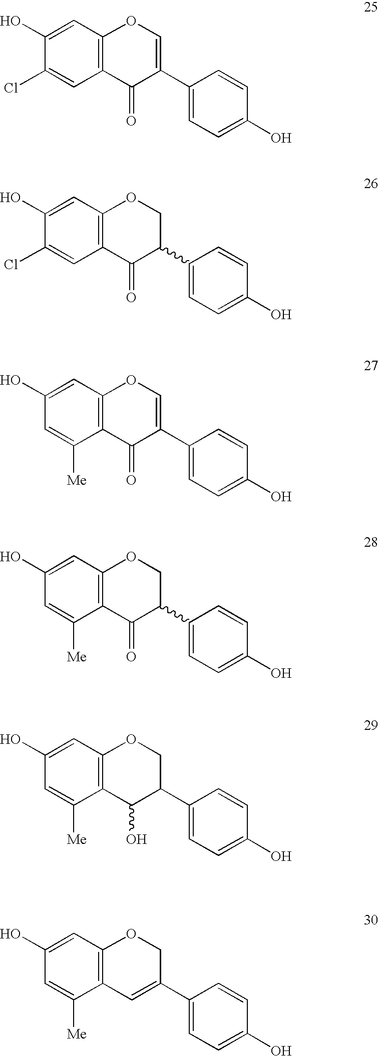

12. The method of claim 11 wherein the isoflavonoid is phenoxodiol (compound 12).

13. A method for treating or preventing nerve damage in a subject, the method comprising administering to the subject an effective amount of an isoflavonoid of formula (I).

14. The method of claim 12 wherein the nerve damage is peripheral nerve damage.

15. The method of claim 13 wherein the nerve damage is induced by, or associated with treatment of the subject with at least one chemotherapeutic agent.

16. The method of claim 13 wherein the isoflavonoid is phenoxodiol.

17. Use of an isoflavonoid of formula (I) as a neuroprotective agent.

18. Use according to claim 17 wherein the isoflavonoid is phenoxodiol.

19. A method for the treatment of cancer in a subject, the method comprising administering to the subject:
(i) a chemotherapeutic agent which has a neurotoxic effect on peripheral nerves, the chemotherapeutic agent being administered at a therapeutically effective dose; and
(ii) an isoflavonoid of formula (I) afa dose effective to prevent, reduce, eliminate or reverse the neurotoxic effect of the chemotherapeutic agent of (i).

20. The method of claim 19 wherein the chemotherapeutic agent and the isoflavonoid are administered concurrently.

21. The method of claim 19 wherein the chemotherapeutic agent and the isoflavonoid are administered sequentially.

22. The method of claim 19 wherein the neurotoxic effect is neuronal dysfunction or damage.

23. The method of claim 19 wherein the isoflavonoid is phenoxodiol.

24. Use of an isoflavonoid of formula (I) for the manufacture of a medicament for the treatment or prevention of neuropathy or a neuropathy-related condition, wherein the neuropathy or neuropathy-related condition is induced by, or otherwise associated with, at least one chemotherapeutic agent.

25. Use of an isoflavonoid of formula (I) for the manufacture of a medicament for the treatment or prevention of nerve damage, wherein the nerve damage is typically induced by, or associated with, a chemotherapeutic agent.

26. Use of an isoflavonoid of formula (I) for the treatment or prevention of neuropathy or a neuropathy-related condition, wherein the neuropathy or neuropathy-related condition is induced by, or otherwise associated with, at least one chemotherapeutic agent.

27. Use of an isoflavonoid of formula (I) for the treatment or prevention of nerve damage, wherein the nerve damage is typically induced by, or associated with, a chemotherapeutic agent.

28. A composition comprising an isoflavonoid of formula (I) when used for the treatment or prevention of neuropathy or a neuropathy-related condition, wherein the neuropathy or neuropathy-related condition is induced by, or otherwise associated with, at least one chemotherapeutic agent.

29. A composition comprising an isoflavonoid of formula (I) when used for the treatment or prevention of nerve damage, wherein the nerve damage is typically induced by, or associated with, a chemotherapeutic agent.
